(19) United States
(12) Patent Application Publication
(10) Pub. No.: US 2025/0032997 A1
(43) Pub. Date: Jan. 30, 2025

(54) LOW POROSITY TANGENTIAL FLOW DEPTH FILTERS

(71) Applicant: Repligen Corporation, Waltham, MA (US)

(72) Inventors: Michael Bransby, Altadena, CA (US); Philip Yuen, Long Beach, CA (US); Derek Carroll, Los Angeles, CA (US)

(73) Assignee: Repligen Corporation, Waltham, MA (US)

(21) Appl. No.: 18/781,201

(22) Filed: Jul. 23, 2024

Related U.S. Application Data
(60) Provisional application No. 63/528,454, filed on Jul. 24, 2023.

(51) Int. Cl. B01D 69/08; B01D 71/26; B01D 71/34; B01D 71/36; C12M 1/00

(52) U.S. Cl. CPC B01D 69/08; B01D 71/261; B01D 71/34; B01D 71/36; C12M 29/16; B01D 2325/0283

(57) ABSTRACT

The present invention relates to hollow fiber tangential flow depth filtration media adapted for perfusion bioprocessing, and related TFDF filtration units, bioprocessing systems, and methods, including methods for perfusion cell culture. The hollow fiber elements of the present invention combine a relatively thin porous wall with a relatively low porosity to provide filtration media resistant to fouling during perfusion culture.

### CROSS-REFERENCE TO RELATED APPLICATIONS

This application claims the benefit of and priority under 35 U.S.C. § 119 to U.S. Provisional Patent Application No. 63/528,454, filed on Jul. 24, 2023, the contents of which are hereby incorporated by reference in their entirety.

### BACKGROUND

Perfusion bioreactors are used in commercial production of cell products including therapeutic proteins, antibodies, and gene therapy vectors such as adeno-associated virus (AAV) vectors and particles. Perfusion bioprocessing describes a cell culture process in which spent media is continuously removed and replaced with fresh media. Perfusion cell culture allows for high density to be attained within the bioreactor while maintaining low levels of waste products, generally improving cell health and viability. Compared to fed-batch processes, for example, an unoptimized perfusion process may achieve up to 10-fold the viable cell density (VCD), providing both higher cell growth rates and increased yield of cell products while maintaining healthier cultures. Tangential flow filtration (TFF) and alternating tangential flow filtration systems (ATF, Repligen, Waltham, MA) have been shown to be robust systems for supporting high cell densities in perfusion cell culture. Nevertheless, perfusion bioreactor systems are compromised by filter fouling and product retention. Product retention refers to the ability of the filter to maintain a desired product concentration in the permeate and retentate streams. This capacity is limited by membrane fouling, which may result in an undesirable increase in product in the retentate stream. Filter fouling refers to a build-up of cells, cell debris, particulate, and extracellular material either at the surface of the filter or within its pores. Improved filtration media, processes, and systems are needed to maintain high filter flux and reduced fouling during perfusion culture operations. The present invention addresses this need.

### BRIEF SUMMARY

The disclosure provides tangential flow depth filters optimized for perfusion cell culture and related systems and methods. In one aspect, a tangential flow depth filtration (TFDF) unit includes one or more hollow fiber (HF) depth filter elements, each including a porous wall having a thickness of from about 1.5-5 millimeters (mm) or about 1.5-3.25 mm defining a lumen with a proximal end including an inlet adapted for fluid communication with an outlet of a process vessel and a distal end including a retentate fluid outlet adapted for fluid communication with an inlet of the process vessel, and a permeate fluid outlet in fluid communication with the porous wall(s) of the HF element(s), where each HF element has a calculated porosity of between about 35-50% or from about 35-45%, or from about 40-45% or from about 44-48% and a normalized water permeability (NWP) of about 1,500-5,000 LMH/psi or about 1,500-3,000 LMH/psi.

The TFDF unit may also include where each HF element has an average pore size of about 0.2 micrometers (μm) up to about 10 μm. In embodiments, the average pore size may be about 0.2, 0.5, 1.0, 2.0, 5.0, 6.0, 7.0, 8.0, 9.0, or 10.0 microns.

The TFDF unit may also include where the porous wall of each HF element has a thickness ranging from about 1.75 mm to about 5 mm or about 1.75 mm to about 3.25 mm or from about 1.75-3 mm, or from about 1.75-2.75 mm.

The TFDF unit may also include where the porous wall of each HF element has a porosity of about 35%, about 40%, about 44%, about 45%, about 46%, about 47%, or about 48%.

The TFDF unit may also include where the one or more tubular HF elements has a thickness of from about 1.75-5 mm or about 1.75-3.25 mm and a porosity of about 35%, 40%, 44%, 45%, 46%, 47%, or 48%. In some aspects one or more tubular HF elements may be from about 2-5 mm thick or from about 2-3 mm thick with a porosity of from about 35-45% or about 44-48%.

The TFDF unit may also include where each HF element is formed from at least one polymer.

The TFDF unit may also include where the wall of each HF element includes a polymer coating.

The TFDF unit may also include where each HF element includes bi-component filaments having a core and a coating.

The TFDF unit may also include where the at least one polymer includes polyethylene (PE), polyethylene terephthalate (PET), polybutylene terephthalate, nylon, polyvinylidene fluoride, polytetrafluoroethylene, or a combination thereof.

The TFDF unit may also include where the polymer coating includes a polymer selected from a polyolefin or a polyethylene.

The TFDF unit may also include where the polymer coating is applied on an outside wall of the HF element, or where the coating is applied to a luminal wall of the HF element.

The TFDF unit may also include where the bi-component filaments are coated with polyethylene (PE), polyethylene terephthalate (PET), polybutylene terephthalate, nylon, polyvinylidene fluoride, polytetrafluoroethylene. Other technical features may be readily apparent to one skilled in the art from the following figures, descriptions, and claims.

In another aspect, the disclosure provides bioprocessing systems and methods for perfusion cell culture. In one aspect, a bioprocessing system includes a process vessel containing a fluid includes cells and a tangential flow depth filtration (TFDF) unit includes one or more tubular hollow fiber (HF) filter elements, each HF element includes a porous wall having a thickness of from about 1.5-5 millimeters (mm) or about 1.5-3.25 mm defining a lumen with a proximal end including an inlet adapted for fluid communication with an outlet of the process vessel and a distal end including a retentate fluid outlet adapted for fluid communication with an inlet of the process vessel, and a permeate fluid outlet in fluid communication with the porous wall(s) of the HF element(s), where each HF element has a porosity of about 30-50% or about 44-48% and a normalized water permeability (NWP) of about 1500-5,000 LMH/psi or about 3000 LMH/psi.

The bioprocessing system may also include where each HF element has an average pore size of about 0.2 micrometers (μm) to about 10 μm.

The bioprocessing system may also include where the porous wall of each HF element has a thickness ranging from about 1.75 mm to about 5 mm or about 1.75 mm to about 3.25 mm or from about 1.75-3 mm, or from about 1.75-2.75 mm.

The bioprocessing system may also include where the porous wall of each HF element has a porosity of about 30-50% or 30-45% or about 44-48%.

The bioprocessing system may also include where each HF element is formed from at least one polymer.

The bioprocessing system may also include where the wall of each HF element includes a polymer coating.

The bioprocessing system may also include where each HF element includes bi-component filaments having a core and a coating. Other technical features may be readily apparent to one skilled in the art from the following figures, descriptions, and claims.

The bioprocessing system may also include where the one or more tubular HF elements has a thickness of from about 1.75-5 mm or about 1.75-3 mm and a porosity of about 30-50% or about 44-48%. The bioprocessing system may also include where the one or more tubular HF elements has a thickness of from about 2-5 mm or about 2-3 mm and a porosity of about 30-45% or about 44-48%.

The bioprocessing system may also include where the at least one polymer includes polyethylene, polyethylene terephthalate, polybutylene terephthalate, nylon, polyvinylidene fluoride, polytetrafluoroethylene, or a combination thereof.

The bioprocessing system may also include where the polymer coating includes a polymer selected from the group consisting of polyolefin, polyethylene, polypropylene (PP), and polyvinylidene fluoride (PVDF).

The bioprocessing system may also include where the polymer coating is applied on an outside wall of the HF element, or on a luminal wall of the HF element.

The bioprocessing system may also include where the bi-component filaments are coated with polyvinylidene fluoride (PVDF).

In one aspect, the disclosure provides a method for perfusion cell culture. In one aspect, the method includes flowing a fluid from a process vessel into a tangential flow depth filter (TFDF) unit as defined in any one of claims 1-12 at an average permeate flow rate of about 800 to 1000 LMH, thereby separating the cell culture fluid into a filtrate and a retentate containing viable cells, and returning the viable cells to the process vessel. Other technical features may be readily apparent to one skilled in the art from the following figures, descriptions, and claims.

### BRIEF DESCRIPTION OF THE SEVERAL VIEWS OF THE DRAWINGS

FIG. 1 shows a graph of permeability (NWP in LMH/psi) versus calculated porosity of series of HF filter elements formed from bi-component filaments of PET/PET.

FIG. 2 shows a bar graph illustrating the effects of wall thickness (5 mm vs 2 mm) on permeability (NWP) for HF filter elements formed from bi-component filaments of 70% polypropylene (PP)/30% polyethylene terephthalate (PET), having a density of 0.53 g/cc and a porosity of 0.49.

FIG. 3A shows a line graph of permeate flux (LMH) versus length of an HF filter element depicting permeate flux (solid line) and fouling flux (dotted line). The graph was generated according to a model of an HF filter element within the scope of the present disclosure and shows operation in non-fouling conditions.

FIG. 3B shows a line graph of permeate flux (LMH) versus length of an HF filter element depicting permeate flux (solid line) and fouling flux (dotted line). The graph was generated according to a model of an HF filter element operating outside the scope of the present disclosure, for comparison to FIG. 3A, and shows fouling conditions.

FIG. 4 illustrates the flow diagram for an experiment comparing the performance of three TFDF units having HF membranes of different porosities in a perfusion cell culture environment.

FIG. 5 is a line graph showing transmembrane pressure (“TMP”, psi) versus volumetric throughput (L/m²) for a standard TFDF membrane (solid line) and two lower porosity membranes, (i) TFDF-7134 (dot-dash line) and (ii) TFDF-7135 (dashed line).

### DETAILED DESCRIPTION

The present invention relates to hollow fiber (“HF”) tangential flow depth filtration media adapted for high efficiency perfusion cell culture operations. Provided are filtration media that are less susceptible to premature fouling and adapted to maintain a flux suitable for high efficiency perfusion. This is accomplished by providing media with lower porosity which reduces or eliminates starling flow that otherwise contributes to premature membrane fouling. In accordance with the present invention, a suitable flux is maintained by providing for additional surface area, e.g., by increasing the number of filter elements or tubes by adapting wall thickness as described herein. In accordance with the methods described here, perfusion operations performed at an average permeate flow rate of about 800 to 1000 LMH through the HF tangential flow depth filtration media are effective to maintain sufficient lift forces to prevent cell adhesion to the membrane without requiring non-laminar or turbulent fluid flow through the filter media. In some aspects, the HF tangential flow depth filtration media has a normalized water permeability (NWP) of about 1000-3000 LMH/psi or about 1500-2500 LMH/psi.

In accordance with some aspects of the methods described here, flow through the lumen of the tangential flow depth filtration media is non-turbulent but maintains sufficient lift forces to prevent fouling during perfusion over prolonged times, from days to weeks. This is in contrast to prior tangential flow depth filtration methods and systems which employed non-laminar feed flows through the filter medium to prevent fouling.

In contrast to prior art tangential flow depth filtration (TFDF) media, the TFDF media of the present invention include a relatively thin porous wall, for example about 1.5-5 millimeters (mm) thick or 1.5-3 mm thick, having a low porosity, for example in a range of from 30-50%, or 44-48%, or 30-45%, or about 30%, 35%, 40%, 44%, 45%, 46% 47% or 48%. In some aspects, the thin-walled, low porosity TFDF membrane has a porosity in the range of about 43-48%, or from about 44-47% or about 45%, about 46%, or about 47%. The low porosity TFDF membranes described herein are less susceptible to premature fouling while maintaining a flux suitable for high efficiency perfusion, e.g., an average permeate flow rate in a range of from about 800 to 1000 LMH, and are characterized by a normalized water permeability (NWP) of about 1000-3000 LMH/psi or about 1500-2500 LMH/psi.

Porosity (P) is calculated as a weight percentage based on the density (d) of the hollow fiber element(s) measured in grams per cubic centimeter (g/cc). Where the hollow fiber element(s) consist of more than one type of polymer, a “dA” term takes into account the aggregate or blended density of the material (dA) such that porosity of the aggregate material is calculated as:

$$P = 1 - ( d / dA ) .$$

For example, where the hollow fiber element(s) are made of bi-component materials, dA is calculated as a sum of each polymer's density multiplied by its weight percentage in the material. Thus, for a material comprised of two polymers, P1 and P2, present in amounts of 70/30 weight percent, respectively, each having a density d1 and d2, respectively, the aggregate density is calculated as

$$dA = ( 0 \text{.70} \times d 1 ) + ( 0.3 \times d 2 ) .$$

“Tangential flow (TF)” refers to fluid flow directed across the surface of a filter medium and as such may also be referred to as “crossflow”. TF is distinguished from “dead end” filtration in which the flow is directed into the surface of a filter medium, and as such may also be referred to as “direct flow” filtration. “Tangential flow filtration” refers to a filtration process in which an inlet fluid stream is divided into two channels, a first channel which flows tangential to the surface of the filter medium, referred to as the retentate fluid channel or retentate flow, and a second channel which enters the filter medium and passes through, referred to as the permeate fluid channel or permeate flow. “Tangential flow depth filtration” or “TFDF” refers to a filtration process that combines tangential flow filtration with filtration through a unique porous wall. “TFDF®” is a registered trademark of Repligen Corp., Waltham MA.

“Hollow fiber element” refers to a filter medium in the form of a hollow fiber comprising a porous wall defining a lumen. Hollow fiber filtration media are generally sealed on opposite ends by a process referred to as “resin potting” in a shell and tube design. In accordance with this general design, the flow path can be from either direction of the tubes to either end of the potting, e.g., from inside to outside or vice versa. The term “hollow fiber” as used herein refers to hollow fiber filtration media or membranes. “Porous wall” refers to the porous wall of a filter medium.

The hollow fiber elements of the TFDF filter media described here are formed from non-woven sintered or melt-blown polymer fibers. The terms “fibers” and “filaments” in the context of “polymer fibers” or “polymer filaments” are used interchangeably herein. Polymers that may be used include polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others. Suitable polyethylene polymers include, high-density polyethylene (HDPE) and high- or ultra-high-molecular weight polyethylene (UHMWPE). In some aspects, the polymer is selected from polypropylene, a polyester, and mixtures thereof.

The term “sintered” in this context refers to the use of heat and optionally pressure in a bonding process. In this process, the polymer fibers are heated to a point where the filaments partially melt and become bonded together at various contact points, optionally, while also compressing the filaments. Thus, sintering bonds fibers where they touch, creating void spaces between the fibers. Numerous fine extruded filaments may be bonded together to at various points to form a hollow fiber, for example, by forming a tubular shape from the extruded filaments and heating the filaments to bond the filaments together.

The term “melt-blown” refers to the use of a gas stream at an exit of a filament extrusion die to attenuate or thin out the filaments while they are in their molten state. Melt-blown filaments are described, for example, in U.S. Pat. No. 5,607,766 to Berger. Mono- or bi-component filaments may be attenuated as they exit an extrusion die using known melt-blowing techniques to produce a collection of filaments. The collection of filaments may then be bonded together in the form of a hollow fiber.

In some aspects, hollow fibers for use in the TFDF filter media described here may be formed by combining bicomponent filaments having a sheath of first material which is bondable at a lower temperature than the melting point of the core material. For example, hollow fibers may be formed by combining bicomponent extrusion technology with melt-blown attenuation to produce a web of entangled biocomponent filaments, and then shaping and heating the web, for example in an oven or using a heated fluid such as steam or heated air, to bond the filaments at their points of contact. An example of a sheath-core melt-blown die is schematically illustrated in U.S. Pat. No. 5,607,766 in which a molten sheath-forming polymer and a molten core-forming polymer are fed into the die and extruded together. The molten bicomponent sheath-core filaments are extruded into a high velocity air stream, which attenuates the filaments, enabling the production of fine bicomponent filaments. U.S. Pat. No. 3,095,343 to Berger shows an apparatus for gathering and heat-treating a multi-filament web to form a continuous tubular body, such as a hollow fiber, of filaments randomly oriented primarily in a longitudinal direction, in which the body of filaments are, as a whole, longitudinally aligned and are, in the aggregate, in a parallel orientation, but which have short portions running at random in non-parallel diverging and converging directions. In this way, a web of sheath-core bicomponent filaments may be pulled into a confined area, for example by using a tapered nozzle having a central passageway forming member, where it is gathered into tubular rod shape and heated or otherwise cured to bond the filaments.

The hollow fiber element(s) of the filter medium does not have a defined pore size due its construction. However, pore size may be determined using methods known in the art, for example a “bubble point test.” The bubble point test is based on the fact that, for a given fluid and pore size, with constant wetting, the pressure required to force an air bubble through a pore is inversely proportional to the pore diameter. In practice, this means that the largest pore size of a filter can be established by wetting the filter material with a fluid and measuring the pressure at which a continuous stream of bubbles is first seen downstream of the wetted filter. The point at which a first stream of bubbles emerges from the filter material is a reflection of the largest pore(s) in the filter material, with the relationship between pressure and pore size being based on Poiseuille's law which can be simplified to P=K/d, where P is the gas pressure at the time of emergence of the stream of bubbles, K is an empirical constant dependent on the filter material, and d is pore diameter. In this regard, pore sizes determined experimentally may be measured using a device such as a POROLUX™ 1000 Porometer (Porometer NV, Belgium), or similar device.

In some aspects, the hollow fiber elements comprising the tangential flow depth filtration media of the present invention combine features of wall porosity and thickness to produce an optimized media for perfusion culture. In some aspects, the filter media comprises a porous wall of from about 1.25 to 5 mm thick or from about 1.25 to 3.5 mm thick or from 1.75 mm to about 3.25 mm thick, or from about 1.75 mm to about 2.75 mm thick, or from about 1.75 mm to about 2.5 mm thick, combined with a porosity of about 30-60% or about 44-48%.

In some aspects, a filter unit for use in the systems and methods described here may comprise one or a plurality of hollow fiber elements, for example 1, 10, 20, 30, 40, or 50 hollow fiber elements. In some aspects, a filter unit for use in the systems and methods described here may comprise a plurality of from about 10-100 or from about 10-50 hollow fiber elements. The number of elements, or tubes, in a filter unit may be selected to accommodate a particular process fluid volume. For example, by increasing the number of tubes, and optionally the length of the tubes, the surface area of the filter media may also be increased. For example, a single HF element may have a surface area of from 1-5 square centimeters (cm²) and be suitable for filtering volumes of less than a liter, or a single HF element may have a surface area of from 100-200 cm² and be suitable for filtering volumes of less than 50 liters. In another example, a filter unit may comprise 10 HF elements with a combined surface area of from about 1000-2000 cm² and be suitable for filtering volumes of less than 500 liters; or a filter unit may comprise 30-50 HF elements with a combined surface area of from about 3000-6000 cm² and be suitable for filtering volumes of less than 2000 liters, or volumes of from about 50-2000 liters.

The particles and/or filaments of the HF filter medium may be formed from any non-woven polymer. In some aspects, the non-woven polymer is selected from a polypropylene, a polyester, and mixtures thereof.

In the context of the methods described here, flow through the filter unit may be a laminar, non-turbulent flow sufficient to create a lift force across the surface of the filter medium that prevents cell from being deposited on the membrane surface. “Filter medium” refers to the medium used to perform a filtration process and in the context of the present invention is in the configuration of a hollow fiber. The terms “filter medium” and “filter membrane” may be used interchangeably herein. Typically the filter medium is disposed within a filter housing which may be, for example, in a tubular form. In embodiments, the filter medium is encapsulated in a filter housing to provide an integral device that may be a single-use or disposable filter unit.

The TFDF filter units described here include a filter housing enclosing the filter medium and directing a fluid flow path through the medium which divides the fluid into retentate and permeate fluid streams. The filter housing is placed in fluid communication with inlet and outlet fluid streams of a process fluid flow. Generally, a filter housing includes a process fluid inlet or inlet port to bring process fluid into the housing at an upstream or proximal end of the filter unit and a retentate outlet or outlet port to bring retentate fluid out of the housing from the downstream or distal end of the filter unit. A filter housing suitable for use with the filter units described here will also include at least one permeate outlet or port to bring the permeate fluid out of the housing. A housing may include other ports, for example a vent port and a drain port. In embodiments, the filter medium is encapsulated in a filter housing to provide an integral device that may be a single-use or disposable filter unit.

“Filter system” refers to a collection of elements for performing fluid filtration and may include, for example, a filter medium encapsulated in a filter housing and in fluid communication with a process vessel containing process fluid, at least one pump, a control system, and associated fittings and connections. In some aspects, a filter system as described herein includes associated fittings and connections to create a recirculating flow path from a process vessel, such as a bioreactor, through a tangential flow depth filtration medium contained in a filter unit as described herein, and back to the bioreactor. The filter system may further comprise fittings and connections to capture a permeate stream of the filter unit

“Filter unit” includes a filtration module as described herein disposed within a filter housing which includes associated ports, for example a fluid inlet port, a retentate outlet port, and at least one permeate outlet port.

“Flux” refers to the flow rate of the filtrate through a filter medium and is generally given in units of volume per unit area and time, such as liters/m²/hour (LMH). In the context of the present disclosure, flux refers to the flow rate of the permeate through the filter medium, typically expressed as an average value. The filter media described here balance wall thickness and porosity to maintain a high fluid flux during perfusion operations that may be expressed in terms of an average permeate flow rate in a range of from about 800 to 1000 LMH.

The tangential flow depth filtration media described here are particularly useful in bioprocessing perfusion applications in which cell debris and cell-produced product, such as a protein product, are continuously removed from cell culture medium as a permeate fluid while viable cells are retained in a retentate fluid and returned to a bioreactor. “Process fluid” refers to a fluid comprising a mixture of cells and/or cell debris and, optionally, a cell product of interest. “Process vessel” refers to a vessel for containing a fluid, for example a suspension culture of cells. The term process vessel includes, for example, a bioreactor.

In some aspects, the hollow fiber elements of the tangential flow depth filtration media described here are melt-blown filaments. As used herein, the term “melt-blown” refers to the use of a gas stream at an exit of a filament extrusion die to attenuate or thin out the filaments while they are in their molten state. Melt-blown filaments are described, for example, in U.S. Pat. No. 5,607,766 to Berger. In various embodiments, mono- or bi-component filaments are attenuated as they exit an extrusion die using known melt-blowing techniques to produce a collection of filaments. The collection of filaments may then be bonded together in the form of a hollow fiber. The pore size and distribution of hollow fibers formed from particles and/or filaments will depend on the size and distribution of the particles and/or filaments that are assembled to form the hollow fibers.

Suitable particles and/or filaments include both inorganic and organic particles and/or filaments. In some embodiments, the particles and/or filaments may be mono-component particles and/or mono-component filaments. In some embodiments, the particles and/or filaments may be multi-component (e.g., bi-component, tri-component, etc.) particles and/or filaments. For example, bi-component particles and/or filaments having a core formed of a first component and a coating or sheath formed of a second component, may be employed, among many other possibilities.

In various embodiments, the particles and/or filaments may be made from polymers. For example, the particles and/or filaments may be polymeric mono-component particles and/or filaments formed from a single polymer, or they may be polymeric multi-component (i.e., bi-component, tri-component, etc.) particles and/or filaments formed from two, three, or more polymers. A variety of polymers may be used to form mono-component and multi-component particles and/or filaments including polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others. Suitable polyethylene polymers include, without limitation, high-density polyethylene (HDPE) and high- or ultra-high-molecular weight polyethylene (UHMWPE)

Particles may be formed into tubular shapes by using, for example, tubular molds. Once formed in a tubular shape, particles may be bonded together using any suitable process. For instance, particles may be bonded together by heating the particles to a point where the particles partially melt and become bonded together at various contact points (a process known as sintering), optionally, while also compressing the particles. As another example, the particles may be bonded together by using a suitable adhesive to bond the particles to one another at various contact points, optionally, while also compressing the particles.

Filament-based fabrication techniques that can be used to form tubular shapes include, for example, simultaneous extrusion (e.g., melt-extrusion, solvent-based extrusion, etc.) from multiple extrusion dies, or electrospinning or electrospraying onto a rod-shaped substrate (which is subsequently removed), among others.

Filaments may be bonded together using any suitable process. For instance, filaments may be bonded together by heating the filaments to a point where the filaments partially melt and become bonded together at various contact points, optionally, while also compressing the filaments. As another example, filaments may be bonded together by using a suitable adhesive to bond the filaments to one another at various contact points, optionally while also compressing the filaments.

In particular embodiments, numerous fine extruded filaments may be bonded together to at various points to form a hollow fiber, for example, by forming a tubular shape from the extruded filaments and heating the filaments to bond the filaments together, among other possibilities.

In certain embodiments, hollow fibers for use in the filter units of the invention may be formed by combining bicomponent filaments having a sheath of first material which is bondable at a lower temperature than the melting point of the core material. For example, hollow fibers may be formed by combining bicomponent extrusion technology with melt-blown attenuation to produce a web of entangled biocomponent filaments, and then shaping and heating the web (e.g., in an oven or using a heated fluid such as steam or heated air) to bond the filaments at their points of contact. An example of a sheath-core melt-blown die is schematically illustrated in U.S. Pat. No. 5,607,766 in which a molten sheath-forming polymer and a molten core-forming polymer are fed into the die and extruded from the same. The molten bicomponent sheath-core filaments are extruded into a high velocity air stream, which attenuates the filaments, enabling the production of fine bicomponent filaments. U.S. Pat. No. 3,095,343 to Berger shows an apparatus for gathering and heat-treating a multi-filament web to form a continuous tubular body (e.g., a hollow fiber) of filaments randomly oriented primarily in a longitudinal direction, in which the body of filaments are, as a whole, longitudinally aligned and are, in the aggregate, in a parallel orientation, but which have short portions running at random in non-parallel diverging and converging directions. In this way, a web of sheath-core bicomponent filaments may be pulled into a confined area (e.g., using a tapered nozzle having a central passageway forming member) where it is gathered into tubular rod shape and heated (or otherwise cured) to bond the filaments.

In certain embodiments, the as-formed hollow fiber may be further coated with a suitable coating material (e.g., PVDF) either on the inside or outside of the fiber, which coating process may also act to reduce the pore size of the hollow fiber, if desired.

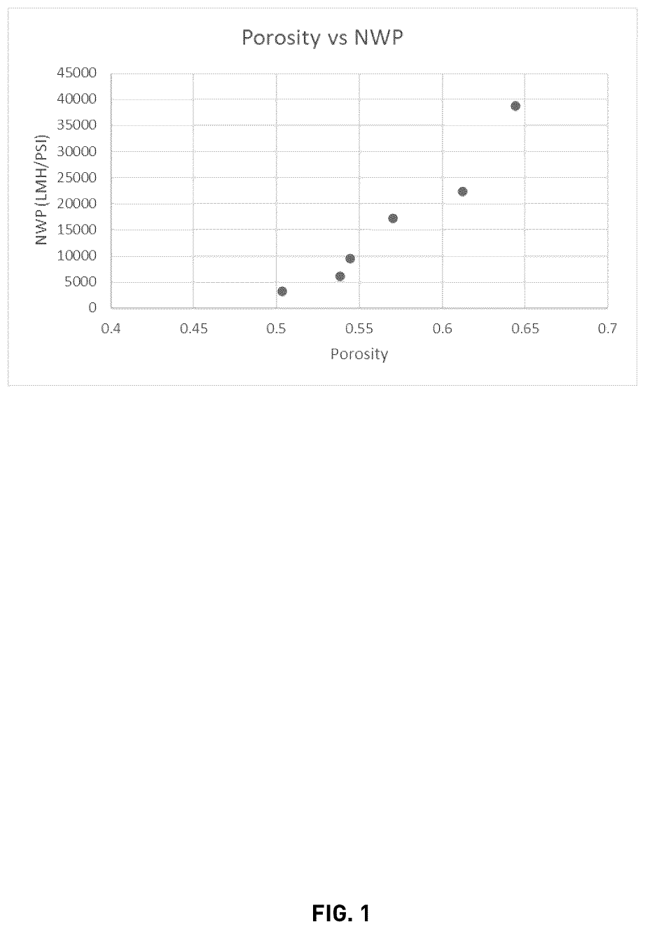

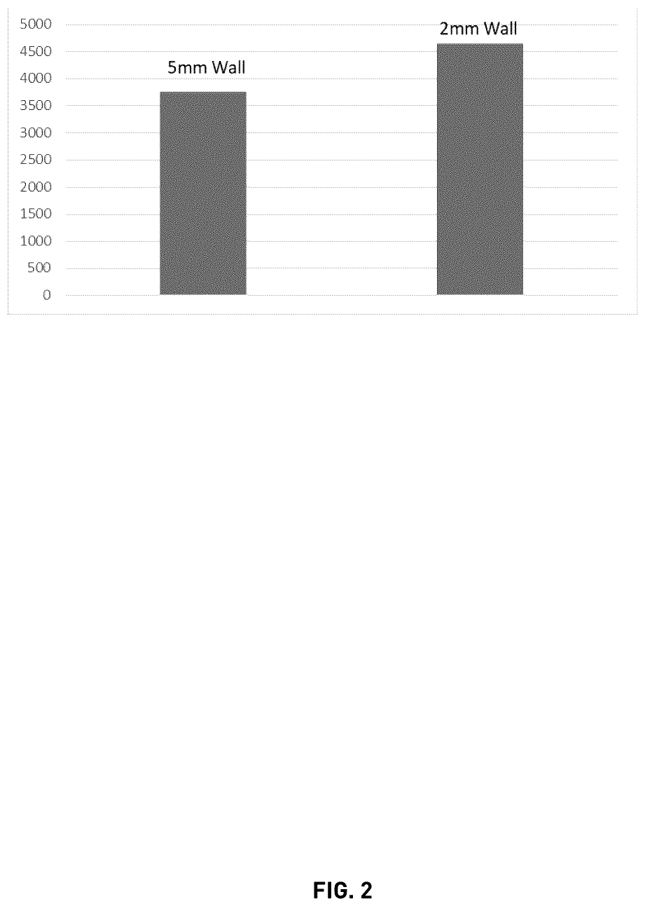

FIG. 1 illustrates data showing that normalized water permeability (NWP) is a function of porosity. HF filter elements were formed from bi-component filaments of polyethylene terephthalate (PET)/PET with 2 mm wall thickness. Porosity is calculated as described in FIG. 2.

Table 1 below gives the porosity and NWP values graphed in FIG. 1, along with the density, porosity, and NWP of six different bi-component membranes constructed of PET/PET 70%.

TABLE 1
Specifications of bi-component PET/PET membranes

| Density (g/cc) | Porosity | NWP |
|---|---|---|
| 0.480 | 0.644 | 38719 |
| 0.523 | 0.613 | 22291 |
| 0.580 | 0.570 | 17173 |
| 0.615 | 0.544 | 9458 |
| 0.623 | 0.539 | 6153 |
| 0.670 | 0.504 | 3144 |

FIG. 2 shows a bar graph illustrating the effects of wall thickness (5 mm vs 2 mm) on permeability (NWP) using reference HF filter elements formed from bi-component filaments of 70% polypropylene (PP)/30% polyethylene terephthalate (PET), having a density of 0.53 g/cc and a calculated porosity of 0.49.

Permeability is measured as normalized water permeability (NWP, L/m2-h-bar or “LMH/psi”), which is water flux (rate of clean water flux through the membrane in liters per membrane area per hour (L/m2-h) divided by the transmembrane pressure.

As discussed above, porosity (P) is calculated as a percentage based on the density (d) of the hollow fiber element(s) measured in grams per cubic centimeter (g/cc) taking into account the aggregate or blended density of the polymer material(s) (dA) making up the element(s):

$$P = 1 - ( d / dA ) .$$

For example, where the hollow fiber element(s) are made of bi-component materials, dA is calculated as a sum of each polymer's density multiplied by its weight percentage in the material. Thus, for a material comprised of two polymers, P1 and P2, present in amounts of 70/30 weight percent, each having a density d1 and d2, the aggregate density is calculated as

$$dA = ( 0 \text{.70} \times d 1 ) + ( 0.3 \times d 2 ) .$$

To illustrate, considering the 70% PP/30% PET hollow fiber elements utilized in the experiment represented by FIG. 2, given that the PP/PET hollow fibers have a density (d) of 0.53 g/cc, the density of PP is 0.9 g/cc (d1) and the density of PET is 1.35 g/cc (d2), then the aggregate density (dA) is calculated as

$$dA = ( 0.7 \times 0.9 ) + ( 0.3 \times 1.35 ) = 1.04 g / cc .$$

Porosity is

$$P = ( 1 - ( 0.53 g / cc ) / 1.04 g / cc ) = 0.49 .$$

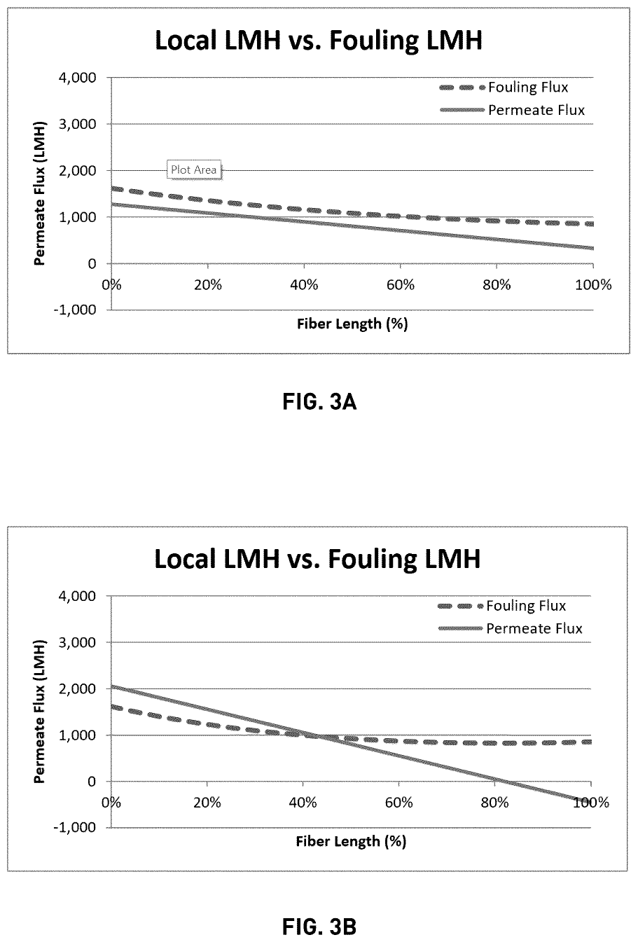

FIG. 3A shows results from a model illustrating operation of a tangential flow depth filtration medium according to the present disclosure where non-fouling conditions are maintained (solid line) across the length of the HF filter element operating within a process flux, of about 400-1200 LMH/psi.

In contrast, FIG. 3B shows results from a comparative model illustrating operation of a reference tangential flow depth filtration medium which results in fouling conditions (dotted line) in the proximal half of the HF filter element.

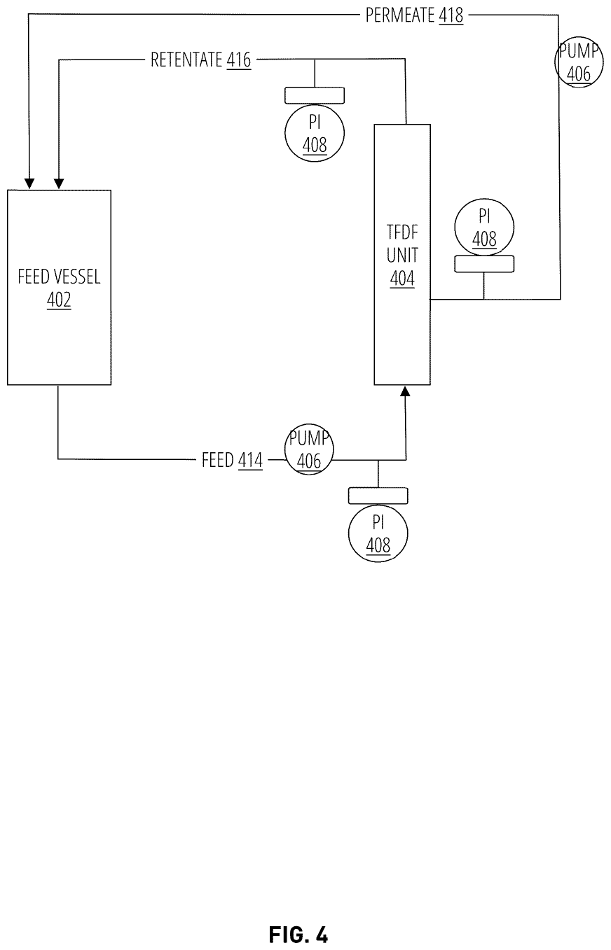

FIG. 4 illustrates the flowpath configuration used in an experiment comparing the performance of three TFDF units having HF membranes of different porosities. Shown are a feed vessel 402 fluidly connected to a TFDF unit 404 along with associated pumps, e.g., pump 406, and pressure sensors on the feed 414, retentate 416, and permeate 418 lines, illustrated as PI 408. The membranes were tested for volumetric throughput of a cell culture fluid containing CHO cells following a 30-day period of perfusion culture. The cell culture fluid contained CHO cells at about 100E6 cells/ml with a cell viability of about 80%. The cells were diluted to about 17E6 cells/ml with perfusion permeate and about 1.6 L of fresh cells was used in each experiment. The TFDF filters were 65 cm in length with a surface area of about 94 cm². During operation, the shear rate was 2100 s⁻¹ (1.2 L/min) and the flux was 1000 LMH (156 ml/min). The membranes were conditioned with perfusion permeate.

All membranes were polypropylene (PP)/polyethylene terephthalate (PET), with the following dimensions: inner diameter 4.6 mm, outer diameter 14.6 mm, wall thickness 5 mm.

The less porous membranes were hypothesized to be able to provide higher throughputs with less fouling compared to more porous materials. This was based in part on the expectation that starling flow would also be decreased with the lower porosity membranes, compared to membranes of higher porosity. With reduced starling flow, the flux should be distributed more evenly over the length of the membrane and the localized permeate flux at the feed side of the filter should be reduced. Under these conditions, the cells and cell debris should not be pushed into the wall of the membrane to the same degree as compared to conditions with higher starling flow, thereby allowing higher volumetric throughputs to be achieved at least in part due to a decrease in fouling.

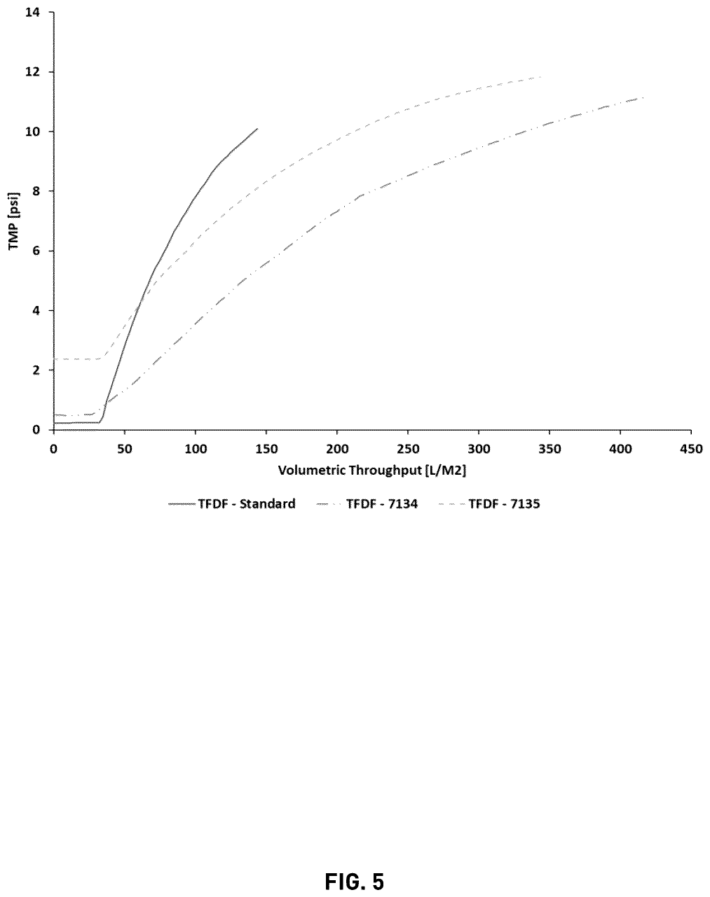

The results of the comparison of performance of TFDF units having HF membranes of different porosities is illustrated in FIG. 5. The figure shows TMP (psi) versus volumetric throughput (L/m²) for a standard TFDF membrane (53% dense, 49% porosity) and two lower porosity membranes, (i) TFDF-7134 (56% dense, 46% porosity) and (ii) TFDF-7135 (60% dense, 42% porosity). The data demonstrate that the standard TFDF membrane (53% dense, 49% porosity) rapidly fouled while both of the lower porosity membranes, TFDF-7134 (56% dense, 46% porosity) and TFDF-7135 (60% dense, 42% porosity) performed better with less fouling and higher volumetric throughput. The best performing membrane was TFDF-7134.

Table 2 below gives the density, porosity, and NWP of the three different bi-component filaments constructed of PP/PET.

TABLE 2
Specifications of bi-component PP/PET membranes

| Membrane | Density (g/cc) | Porosity | NWP |
|---|---|---|---|
| standard TFDF | 1.04 | 0.490 | 3760 |
| TFDF-7134 | 1.04 | 0.461 | 1560 |
| TFDF-7135 | 1.04 | 0.423 | 375 |

### Additional Embodiments

Embodiment 1: A tangential flow depth filtration (TFDF) unit comprising one or more tubular hollow fiber (HF) filter elements, each HF element comprising a porous wall having a thickness of from about 1.5-5 millimeters (mm), optionally about 1.5-3.25 mm, defining a lumen with a proximal end including an inlet adapted for fluid communication with an outlet of a process vessel and a distal end including a retentate fluid outlet adapted for fluid communication with an inlet of the process vessel, and a permeate fluid outlet in fluid communication with the porous wall(s) of the HF element(s); wherein each HF element has a porosity between about 30%-50%.

Embodiment 2: The TFDF unit of embodiment 1, wherein each HF element has a porosity between about 44-48%.

Embodiment 3: The TFDF unit of embodiment 1 or 2, wherein each HF element has an average pore size of about 0.2 micrometers (μm) to about 10 μm.

Embodiment 4: The TFDF unit of any one of embodiments 1 to 3, wherein the porous wall of each HF element has a thickness ranging from about 1.75 mm to about 5 mm, about 1.75 mm to about 3.25 mm or from about 1.75-3 mm, or from about 1.75-2.75 mm.

Embodiment 5: The TFDF unit of any one of embodiments 1 to 4, wherein the porous wall of each HF element has a porosity of about 30-45% or about 44-48%.

Embodiment 6: The TFDF unit of any one of embodiments 1 to 5, wherein the one or more tubular HF elements has a thickness of from about 1.75 mm to about 5 mm, or about 1.75 mm to about 3 mm, and a porosity of about 30-45% or about 44-48%.

Embodiment 7: The TFDF unit of any one of embodiments 1 to 6, wherein each HF element is formed from at least one polymer.

Embodiment 8: The TFDF unit of embodiment 7, wherein the at least one polymer comprises polyethylene, polyethylene terephthalate, polybutylene terephthalate, nylon, polyvinylidene fluoride, polytetrafluoroethylene, or a combination thereof.

Embodiment 9: The TFDF unit of any one of embodiments 1 to 8, wherein the wall of each HF element comprises a polymer coating.

Embodiment 10: The TFDF unit of embodiment 9, wherein the polymer coating comprises a polymer selected from the group consisting of polyolefin, polyethylene, polypropylene, and polyvinylidene fluoride (PVDF).

Embodiment 11: The TFDF unit of embodiment 9 or embodiment 10, wherein the polymer coating is applied on an outside wall of the HF element, or on a luminal wall of the HF element.

Embodiment 12: The TFDF unit of any one of embodiments 1 to 11, wherein each HF element comprises bi-component filaments having a core and a coating.

Embodiment 13: The TFDF unit of embodiment 12, wherein the bi-component filaments are coated with polyvinylidene fluoride (PVDF).

Embodiment 14: A bioprocessing system comprising: a process vessel containing a fluid comprising cells and a tangential flow depth filtration (TFDF) unit comprising one or more tubular hollow fiber (HF) filter elements, each HF element comprising a porous wall having a thickness of from about 1.5-5 millimeters (mm) or from about 1.5-3.25 mm defining a lumen with a proximal end including an inlet adapted for fluid communication with an outlet of the process vessel and a distal end including a retentate fluid outlet adapted for fluid communication with an inlet of the process vessel, and a permeate fluid outlet in fluid communication with the porous wall(s) of the HF element(s); wherein each HF element has a porosity of about 30-50% or about 44-48%.

Embodiment 15: The bioprocessing system of embodiment 14, wherein each HF element has an average pore size of about 0.2 micrometers (μm) to about 10 μm.

Embodiment 16: The bioprocessing system of embodiment 14 or embodiment 15, wherein the porous wall of each HF element has a thickness ranging from about 1.75 mm to about 5 mm, or from about 1.75 to 3.25 mm, or from about 1.75 to 2.75 mm.

Embodiment 17: The bioprocessing system of any one of embodiments 14 to 16, wherein the porous wall of each HF element has a porosity of about 30-50% or about 44-48%.

Embodiment 18: The bioprocessing system of embodiment 17, wherein the one or more tubular HF elements has a thickness of from about 1.75-5 mm or about 1.75-2.75 mm and a porosity of about 30-50% or about 44-48%.

Embodiment 19: The bioprocessing system of any one of embodiments 14 to 18, wherein each HF element is formed from at least one polymer.

Embodiment 20: The bioprocessing system of embodiment 19, wherein the at least one polymer comprises polyethylene, polyethylene terephthalate, polybutylene terephthalate, nylon, polyvinylidene fluoride, polytetrafluoroethylene, or a combination thereof.

Embodiment 21: The bioprocessing system of any one of embodiments 14 to 20, wherein the wall of each HF element comprises a polymer coating.

Embodiment 22: The bioprocessing system of embodiment 21, wherein the polymer coating comprises a polymer selected from the group consisting of polyolefin, polyethylene, polypropylene, and polyvinylidene fluoride (PVDF).

Embodiment 23: The bioprocessing system of embodiment 21 or embodiment 22, wherein the polymer coating is applied on an outside wall of the HF element, or on a luminal wall of the HF element.

Embodiment 24: The bioprocessing system of any one of embodiments 14 to 23, wherein each HF element comprises bi-component filaments having a core and a coating.

Embodiment 25: The bioprocessing system of any one of embodiments 14 to 23, wherein each HF element comprises bi-component filaments having a core and a coating.

Embodiment 26: A method for perfusion cell culture, the method comprising: flowing a fluid from a process vessel into a tangential flow depth filter (TFDF) unit as defined in any one of any one of embodiments 1 to 13 at an average permeate flow rate of about 800 to 1000 LMH, thereby separating the cell culture fluid into a filtrate and a retentate containing viable cells; and returning the viable cells to the process vessel.

While the invention herein disclosed has been described by means of various aspects, specific embodiments and applications thereof, numerous modifications and variations could be made thereto by those skilled in the art without departing from the scope of the invention set forth in the claims.

It will be appreciated that the present invention is set forth in various levels of detail in this application. In certain instances, details that are not necessary for one of ordinary skill in the art to understand the invention, or that render other details difficult to perceive may have been omitted. The terminology used herein is for the purpose of describing particular embodiments only and is not intended to be limiting beyond the scope of the appended claims. Unless defined otherwise, technical terms used herein are to be understood as commonly understood by one of ordinary skill in the art to which the disclosure belongs.

Various features of a process system may be used independently of, or in combination, with each other. It will be appreciated that a system as disclosed herein may be embodied in different forms and should not be construed as limited to the illustrated embodiments of the figures.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The term “about” refers to a range of 2-3% around the stated value. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tangential flow depth filtration (TFDF) unit comprising
one or more tubular hollow fiber (HF) filter elements, each HF element comprising a porous wall having a thickness of from about 1.5-5 millimeters (mm), optionally about 1.5-3.25 mm, defining a lumen with a proximal end including an inlet adapted for fluid communication with an outlet of a process vessel and a distal end including a retentate fluid outlet adapted for fluid communication with an inlet of the process vessel, and a permeate fluid outlet in fluid communication with the porous wall(s) of the HF element(s);
wherein each HF element has a porosity between about 30%-50%.

2. The TFDF unit of claim 1, wherein each HF element has a porosity between about 44-48%.

3. The TFDF unit of claim 1, wherein each HF element has an average pore size of about 0.2 micrometers (μm) to about 10 μm.

4. The TFDF unit of claim 1, wherein the porous wall of each HF element has a thickness ranging from about 1.75 mm to about 5 mm, about 1.75 mm to about 3.25 mm or from about 1.75-3 mm, or from about 1.75-2.75 mm.

5. The TFDF unit of claim 1, wherein the one or more tubular HF elements has a thickness of from about 1.75 mm to about 5 mm and a porosity of about 44-48%.

6. The TFDF unit of claim 1, wherein each HF element is formed from at least one polymer comprising polyethylene, polyethylene terephthalate, polybutylene terephthalate, nylon, polyvinylidene fluoride, polytetrafluoroethylene, or a combination thereof.

7. The TFDF unit of claim 1, wherein the wall of each HF element comprises a polymer coating, optionally wherein the polymer coating comprises a polymer selected from the group consisting of polyolefin, polyethylene, polypropylene, and polyvinylidene fluoride (PVDF).

8. The TFDF unit of claim 7, wherein the polymer coating is applied on an outside wall of the HF element, or on a luminal wall of the HF element.

9. The TFDF unit of claim 1, wherein each HF element comprises bi-component filaments having a core and a coating.

10. The TFDF unit of claim 9, wherein the bi-component filaments are coated with polyvinylidene fluoride (PVDF).

11. A bioprocessing system comprising:
a process vessel containing a fluid comprising cells and
a tangential flow depth filtration (TFDF) unit comprising one or more tubular hollow fiber (HF) filter elements, each HF element comprising a porous wall having a thickness of from about 1.5-5 millimeters (mm) defining a lumen with a proximal end including an inlet adapted for fluid communication with an outlet of the process vessel and a distal end including a retentate fluid outlet adapted for fluid communication with an inlet of the process vessel, and a permeate fluid outlet in fluid communication with the porous wall(s) of the HF element(s);
wherein each HF element has a porosity of about 30-50%.

12. The bioprocessing system of claim 11, wherein each HF element has an average pore size of about 0.2 micrometers (μm) to about 10 μm.

13. The bioprocessing system of claim 11, wherein the porous wall of each HF element has a thickness ranging from about 1.75 mm to about 5 mm.

14. The bioprocessing system of claim 11, wherein each HF element has a porosity of about 44-48%.

15. The bioprocessing system of claim 11, wherein the one or more tubular HF elements has a thickness of from about 1.75-5 mm.

16. The bioprocessing system of claim 11, wherein each HF element is formed from at least one polymer.

17. The bioprocessing system of claim 16, wherein the at least one polymer comprises polyethylene, polyethylene terephthalate, polybutylene terephthalate, nylon, polyvinylidene fluoride, polytetrafluoroethylene, or a combination thereof.

18. The bioprocessing system of claim 11, wherein the wall of each HF element comprises a polymer coating.

19. The bioprocessing system of claim 18, wherein the polymer coating comprises a polymer selected from the group consisting of polyolefin, polyethylene, polypropylene, and polyvinylidene fluoride (PVDF).

20. The bioprocessing system of claim 19, wherein the polymer coating is applied on an outside wall of the HF element, or on a luminal wall of the HF element.

21. The bioprocessing system of claim 11, wherein each HF element comprises bi-component filaments having a core and a coating.

22. The bioprocessing system of claim 21, wherein the bi-component filaments are coated with polyvinylidene fluoride (PVDF).

23. A method for perfusion cell culture, the method comprising:
flowing a fluid from a process vessel into a tangential flow depth filter (TFDF) unit at an average permeate flow rate of about 800 to 1000 LMH, thereby separating the cell culture fluid into a filtrate and a retentate containing viable cells; and
returning the viable cells to the process vessel,
wherein the TFDF unit comprises one or more tubular hollow fiber (HF) filter elements, each HF element comprising a porous wall having a thickness of from about 1.5-5 millimeters (mm), optionally about 1.5-3.25 mm, defining a lumen with a proximal end including an inlet adapted for fluid communication with an outlet of a process vessel and a distal end including a retentate fluid outlet adapted for fluid communication with an inlet of the process vessel, and a permeate fluid outlet in fluid communication with the porous wall(s) of the HF element(s); and wherein each HF element has a porosity between about 30%-50%.
